(19) United States
(12) Patent Application Publication
(10) Pub. No.: US 2008/0145420 A1
(43) Pub. Date: Jun. 19, 2008

(54) HUMAN SECRETORY IgA FOR THE TREATMENT OF CLOSTRIDIUM DIFFICILE ASSOCIATED DISEASES

(76) Inventor: Michael R. Simon, Ann Arbor, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/610,154

(22) Filed: Dec. 13, 2006

(51) Int. Cl. A61K 9/54; A61K 39/44; A61P 31/04; A61K 9/28

(52) U.S. Cl. 424/463; 424/178.1; 424/474

(57) ABSTRACT

A composition for treating a subject is provided. The composition includes dimeric or polymeric IgA therapeutic. Formulating agents are mixed with the dimeric or polymeric IgA to yield a dosing form of a capsule, tablet, and a suppository. A process for manufacturing a medicament for the treatment of C. difficile associated disease in a human is also provided that the sequential modification of monomeric IgA with J chain and secretory component to form a dimeric or polymeric IgA therapeutic. The dimeric or polymeric IgA therapeutic is then mixed with formulating agents to create a capsule, tablet, or suppository dosing form. The therapeutic is amenable to enrobement directly through microeneapsulation or the dosing form is coated with an enteric coating. A method of C. difficile treatment with the therapeutic is also provided that is amenable to supplementation with concurrent or prior antibiotic administration.

### FIELD OF THE INVENTION

This invention relates in general to compositions for the treatment of Clostridium difficile associated diseases such as Clostridium difficile colitis, pseudomembranous colitis and antibiotic associated diarrhea and in particular to secretory immunoglobulin A (IgA) compositions administered in the form of pharmaceutical compositions.

### BACKGROUND OF THE INVENTION

Clostridium difficile (C. difficile) is a gram-positive anaerobic bacillus.

Antibiotic associated pseudomembranous colitis results from the use of broad-spectrum antibiotic agents such as clindamycin. These antibiotics cause diarrhea in about 10% of treated patients and pseudomembranous colitis in about 1%. C. difficile causes antibiotic associated diarrhea and almost all cases of pseudomembranous colitis.

Pseudomembranous colitis results from the production of C. difficile toxin A (MW, 308,000) and toxin B (MW, 270,000) in the colon (Barroso et al., Nucleic Acids Res., 18:4004; Dove et al., Infect. Immun., 58:480-488; Lyerly et al., Clin. Microbiol. Rev., 1:1-18). Toxin A probably causes most of the gastrointestinal symptoms because of its enterotoxic activity (Lyerly et al., Infect. Immun., 35:1147-1150; Lyerly et al., Infect. Immun., 47:349-352). The toxins may act synergistically and the initial pathology caused by toxin A allows toxin B to manifest its toxicity (Lyerly et al., Infect. Immun., 47:349-352).

Most patients with C. difficile associated disease are treated effectively with vancomycin or metronidazole. Other treatment modalities include tolevemer, a toxin binding polymer (T. J. Louie et al., Clin. Infect. Dis. 2006; 43:411), and an antiparasitic medication, nitazoxanide (Med. Letter Drugs Ther. 2006; 48:89). However, relapses occur in about 20-25% of patients. Therefore, there is still a need for additional effective treatment of Clostridium difficile associated disease in humans.

Immunological treatment is valuable. Vaccination against toxins A and B stimulates active immunity against C. difficile disease in animals (Libby et al., Infect. Immun., 36:822-829). However, vaccines against the organism and its toxins are not available for human use.

Passive immunization is another immunological treatment. Serum antibodies against C. difficile protect hamsters against C. difficile disease after oral administration. Passive immunization with bovine antibodies has been proposed as a treatment for other infectious diseases of the gastrointestinal tract, such as diseases caused by rotavirus, enteropathogenic and enterotoxigenic Escherichia coli, Vibrio cholerae, and Cryptosporidium parvum. Preliminary studies indicate that such passive immunization provides protection (Boesman-Finkelstein et al., Infect. Immun., 57:1227-1234; Brussow et al., J. Clin. Microbiol., 25:982-986; Fayer et al., Infect. Immun., 58:2962-2965; Hilpert et al., J. Infect. Dis., 156:158-166; Mietens et al., Eur. J. Pediatr., 132:239-252; Tacket et al., N. Engl. J. Med., 318:1240-1243; Yoshiyama et al., Immunology, 61:543-547).

It has been reported that bovine immunoglobulin G (IgG) concentrate from the colostrum of cows vaccinated with C. difficile toxoid protects hamsters against antibiotic associated cecitis. The hamsters were protected when treated before the onset of diarrhea but not after diarrhea began (Lyerly et al,, Infection and Immunity, Vol. 59, No. 6, pages 2215-2218 (1991)).

IgG directed against toxins A and B of C. difficile are present in the general population (Bacon and Fekety, Diagn. Microbiol. Infect. Dis., 1994; 18:205-209). Human intravenous irrunoglobulin derived from plasma donors has facilitated treatment in some patients, especially patients who lack circulating antibodies to the C. difficile toxins (Leung D. Y., et al. J. Pediatr. 1991 April; 118(4(Pt 1)):633-7; Salcedo J. et al., Gut 1997; 41:366-370; Wilcox M H. J. Antimicrob. Chemoth. 2004; 53:882-884; McPherson S,et al. DisColon Rectum. 2006; 49:640-645; Cone L A, et al. Infect Dis Clin Pract 2006 ;14:217-220).

### Paragraph Break Needed Here

In vitro experiments have demonstrated that polymeric IgA is superior to monomeric IgA and IgG in preventing C. difficile toxin damage to intestinal epithelial cell monolayers (Stubbe H. et al., J. Immunol. 2000; 164:1952-1960). Selective neutralization of C. difficile toxin by serum IgA has also been demonstrated (Johnson S. et al., Infect. Immun. 1995; 63:3166-3173).

Administration of an immunoglobulin product containing specific antibodies to C. difficile results in the elimination of C. difficile toxins and also killing of the bacteria within the colon as detailed in U.S. Pat. No. 5,773,000. Such passive immunization therefore provides an effective approach for the treatment of C. difficile associated diseases such as colitis, pseudomembranous colitis and antibiotic associated diarrhea. This is especially important for patients experiencing multiple relapses.

Current treatments for C. difficile associated disease use antibiotics such as metonidazole and vancomycin. These drugs result in further disruption of the intestinal flora and are associated with a 20-25% incidence of disease relapse.

Monomeric Id admixed with IgG (2:1) was derived from plasma (IgAbulin, Immuno, Vienna) (100 mg/mL). Four mL was administered orally 3 times daily for 3 weeks to a three and one-half year old child with antibiotic-associated diarrhea and C. difficile toxin A in his stools. Vancomycin administration was continued concurrently. The child improved on this treatment (Tjellstrom B. et al. Lancet 1993;341:701702). This report demonstrates the efficacy of passive immunization with IgA derived from the general population. The present invention is superior to monomeric IgA administered orally because the presence of secretory component protects the IgA from digestion in the gastrointestinal tract. It appears that monomeric IgA possesses efficacy. However, increased efficacy is achieved by secretory IgA owing to the propensity of monomeric IgA to degrade in the gastrointestinal tract. The resultant dosing requirements increase treatment costs. The prior art use of monomeric IgA failed to explore secretory IgA as a potential medicament.

Thus, there exists a need for an IgA therapeutic that is resistant to gastrointestinal tract degradation. There also exists a need to provide such a therapeutic in a dosing form well suited for treating an infected subject.

### SUMMARY OF THE INVENTION

A composition for treating a subject, especially a human subject, is provided. The composition includes a dimeric or polymeric IgA therapeutic that is formed by combining polyclonal monomeric IgA with a recombinant J chain to form an IgA-J chain conjugate in a molar ratio of the IgA to the J chain of 2:1 or greater and in turn combining the conjugate with a recombinant secretory component in a molar ratio of an IgA-J chain conjugate to the secretory component of 1:1. Formulating agents are mixed with the dimeric or polymeric IgA to yield a dosing form of a capsule, tablet, and a suppository. The IgA therapeutic is optional enterically coated or microencapsulated to withstand gastrointestinal exposure associated with oral delivery. The dosing form is in a daily amount of between 0.1 and 50 grams. The dosing form containing the IgA therapeutic optionally also includes an antibiotic.

A process for manufacturing a medicament for the treatment of C. difficile associated disease in a human is also provided that includes the collection of monomeric IgA as a byproduct of cold ethanol fractionation of pooled plasma derived from more than one human individual or production of monoclonal monomeric IgA by hybridoma technique. The polyclonal monomeric IgA is subjected to antiviral treatment to yield a virus free polyclonal monomeric IgA that is also sterilized. The monomeric IgA regardless of origin is sequentially modified with J chain and secretory component to form a dimeric or polymeric IgA therapeutic. The dimeric or polymeric IgA therapeutic is then mixed with formulating agents to create a capsule, tablet, or suppository dosing form. The pooled plasma is optionally derived from specifically immune or immunized donors. The therapeutic is amenable to enrobement directly through microencapsulation or the dosing form is coated with an enteric coating. A method of treatment for C. difficile with the therapeutic is also provided. The treatment is amenable to supplementation with concurrent or prior antibiotic administration.

### DETAILED DESCRIPTION OF THE PREFERRED EMBODIMENTS

The present invention has utility as a treatment for C. difficile infections. Unlike prior usage of monomeric IgA that is susceptible to gastrointestinal degradation, the present invention uses dimeric- and polymeric- secretory IgA. Because of its resistance to degradation in the gastrointestinal tract, it can be used at lower doses. Dimeric- and polymeric-IgA according to the present invention are bound to I-chain and secretory component in order to mimic secretory IgA endogenous to the subject.

As used herein, a “subject” is defined as a mammal and illustratively includes humans, non-human primates, horses, goats, cows, sheep, pigs, dogs, cats, and rodents.

As the present invention uses an immunoglobulin rather than antibiotics, an effective treatment is provided which does not disturb the intestinal flora.

The following description of the preferred embodiment(s) is merely exemplary in nature and is in no way intended to limit the invention, its application, or uses.

In one embodiment, the invention provides a method for medical treatment of humans involving the oral administration of an secretory IgA component which can be derived from a number of sources. One such source for the IgA is pooled human plasma following Cohn cold ethanol fractionation to produce fraction III precipitate as performed by those of skill in the art of protein separation. IgA byproduct is further purified by adsorption onto an ion exchange medium in neutral or slightly acidic conditions as performed by those of skill in the art of protein purification.

A more detailed description of isolation of an IgA component as a byproduct from pooled human plasma or hyperimmune pooled human plasma is as follows. Ethanol fractionation of pooled human plasma is a well-known process to prepare immunoglobulin G. Pooled human plasma is first obtained from licensed plasmapheresis centers in the United States and tested for various pathogens including the HIV virus. The first manufacturing step of most commercial immunoglobulin G preparations involves a modified cold ethanol fractionation according to Cohn to produce Cohn fraction II. In the fractionation process, many infectious viruses are eliminated from the pooled human plasma. Following fractionation, the Cohn fraction II is subjected to adsorption onto an ion exchange medium. This step may selectively reduce the IgA concentration to less than 0.1%. Such a step is important for producing immunoglobulin G for intravenous infusion into humans. This is because some individuals undergo an anaphylactic-like reaction if treated with intravenous IgG that contains IgA as an impurity.

The modified cold ethanol fractionation process according to Cohn is a series of fractionations using various levels of ethanol, pH, and temperature to produce a fraction II which is further treated to produce immunoglobulins as described above. In the fractionation method, pooled human plasma is first treated to produce a cryoprecipitate and cryo-supernatant. The cryo-supernatant is subjected to a first ethanol fractionation to yield a supernatant I. Supernatant I is subjected to a second ethanol fractionation to yield fraction II+III. Fraction II+III is subjected to a third ethanol fractionation procedure to yield a supernatant III and Fraction III precipitate.

The fraction III precipitate enriched in IgA is generally discarded as an unwanted byproduct. According to the invention, this unwanted IgA following ion exchange adsorption purification is further treated by incubation with immobilized hydrolases to inactivate viruses and vasoactive substances. Such treatment has been proven to eliminate many viruses tested including HIV, Sindbis, and vaccinia. Following incubation to remove viruses, the concentration of the active material is adjusted with sterile saline or buffered solutions to ensure a constant amount of active material per milliliter of reconstituted product. Finally, the solution with a constant amount of reconstituted product is sterilized by filtration before use.

The ethanol fractionation process according to Coin is well known in the art and is described in Cohn et al., J. Am. Chem. Soc. 1946; 68:459-475, Oncley et al., J. Am. Chem. Soc. 1949; 71:541-550, and in most detail in pages 576-602, Kirk-Othmer Encyclopedia of Chemical Technology, Vol. 3, second edition (1963).

In a preferred embodiment, the compositions of the invention contain, in addition to the IgA component, one or more further components selected from the group consisting of recombinant human J chains, recombinant secretory component, and combinations thereof. The production of human J chains by genetically recombinant biological techniques is disclosed in Symerski et al., Mol. Immunol. 2000; 37:133-140. Human secretory component can be produced by recombinant techniques as described in Crottet et al., Biochem. J. 1999; 341:299-306. In a preferred embodiment the IgA may be coupled to recombinant J chains by disulfide bonding which is accomplished in mildly oxidizing conditions. The resulting IgA-J chain conjugates are purified. IGA-J chain conjugates are optionally further coupled to recombinant secretory component. In a preferred embodiment, the coupling is accomplished by forming disulfide bonds under mildly oxidizing conditions. (Jones R. M. L., Schweikart F., Frutiger S., Jaton J-C., Hughes G. J. Thiol-disulfide redox buffers maintain a structure of immunoglobulin A that is essential for optimal in vitro binding to secretory component. Biochimica et Biophysica Acta 1998; 1429:265-274.) IgA containing both J chain and secretory component is again purified by ion-exchange and size exclusion chromatography and/or ultrafiltration as described in Lullau et al., J. Biol. Chem. 1996; 271:16300-16309, Corthesy, Biochem. Soc. Trans. 1997; 25:471-475, and Crottet et al., Biochem. J. 1999; 341:299-306, as performed by those of skill in the art of protein purification. While recombinant expression of IgA with the incorporation of J chain and secretory component has been accomplished, hybridoma production of IgA may not include incorporated J chains together with secretory component. According to the invention, the recombinant J chains, recombinant secretory component, or mixtures thereof are combined with the monoclonal IgA after production of the IgA by hybridoma techniques. Such IgA may be coupled to recombinant J chains and secretory component as described above. Purified IgA containing J chain and secretory components is optionally stabilized for example by the addition of human serum albumin to a final concentration of 5%. The presence of the human J chais and secretory component in the compositions of the invention leads to doses of immunoglobulin A which are more physiologically effective than compositions without such components.

In another embodiment, an IgA containing component is isolated as a byproduct from hyperimmune pooled human plasma for coupling with J chain and secretory component. Hyperimmune pooled human plasma is obtained from donors who have been immunized against a specific disease or are immune to the disease following natural infection.

In another embodiment, the IgA component can be prepared by hybridoma techniques to provide antigen-specific IgA. Hybridoma techniques are described originally in Kohler and Milstein, Nature 1975; 256:495-497 with more recent advances summarized in Berzofsky et al., Fundamental Immunology, Third Edition, 1993, pp 455-462. Hybridoma production involves the fusion of an immortalized immunoglobulin-producing mycloma cell with an antibody producing cell from an immunized individual. The product is an immortalized cell culture which produces the specific antibody against the antigen that the donor individual is immune to. For example, a mouse monoclonal IgA antibody has been prepared against respiratory syncytial virus F glycoprotein as described in Weltzin et al., J. Infect. Dis. 1996; 174:256-261 and Weltzin et al., Antimicrob. Agents Chemother. 1994; 38:2785-2791.

Dimeric and polymeric IgA is prepared with two, and with more than two, IgA monomers per J chain, respectively.

The secretory IgA antibodies may be administered alone as a liquid or solid, preferably in a solid powder form and preferably in admixture with a carrier to form a pharmaceutical composition such as a tablet, capsule or suppository.

Since preferred methods of administration are oral and rectal, or enteric installation, and most preferred is oral, with solid oral dosage forms such as tablets and capsules being especially preferred, or enteric installation. These are prepared according to conventional methods known those skilled in the art. The secretory IgA antibodies may also be combined with other pharmaceutically acceptable carriers such as various liquids, proteins or oils which may also provide additional nutritional and/or pharmaceutical benefits. Remington Science and Practice of Pharmacy, 20^{th} ed. (2000).

These compositions optionally contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and the like. Prolonged absorption of the IgA can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient (or carer) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (i) absorption accelerators, as for example, quaternary ammonium compounds, (g) wetting agents, as for example, cetyl alcohol, and glycerol monostearate, (hi adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well known in the art; as detailed, for example in U.S. Pat. Nos. 4,017,647; 4,385,078; 4,518,433; and 4,556,552.

They may contain opacifying agents, and can also be of such composition that they release the active compound or compounds in a certain part of the intestinal tract in a delayed manner. Examples of embedding compositions which can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacantli, or mixtures of these substances, and the like.

Compositions for rectal administrations are preferably suppositories which can be prepared by mixing the compounds of the present invention with suitable non-irritating excipients or carriers such as cocoa butter, polyethyleneglycol or a suppository wax, which are solid at ordinary temperatures but liquid at body temperature and therefore melt in the rectum or vaginal cavity and release the active component.

Since the effect of the secretory IgA antibody is dependent on its reaching the colons preferred tablets or capsules are enteric coated. Alternatively, the active secretory IgA antibodies can themselves be microencapsulated prior to formulation. Preparation of microcapsules of secretory IgA antibody as well as preparation of enteric coated tablets or capsules can be achieved by conventional methods as detailed above.

Because the secretory IgA antibodies first eliminate the C. difficile toxins, it is also advantageous to administer to patients suffering from C. difficile associated diseases a combination of the secretory IgA antibodies of the present invention with antibiotics that are known for treating pseudomembranous colitis and/or antibiotic associated diarrhea. Such antibiotics are for example vancomycin, and metronidazole. Because of the prompt elimination of the C. difficile toxins, the combination of secretory IgA antibody and antibiotic may be synergistic requiring a shorter duration of antibiotic treatment with decreased symptoms, faster symptomatic relief and a lower relapse rate. Recognized doses for administering metronidazole for example is 250 mg four times a day, and oral vancomycin is 125 mg four times a day. Administration of these antibiotics with the secretory IgA antibody of the present invention would result in use of substantially reduced dosage of antibiotics.

The administration of such combination antibiotic and secretory IgA treatment may be in a single dosage form where both active ingredients are combined and mixed with a pharmaceutically acceptable carrier, Preferred compositions would be those adapted for oral or rectal administration and it would include solid oral dosing forms such as enteric coated tablets or capsules, or suppositories.

The administration of the combination concurrently or following one another in separate dosage forms may still be formulated together in divided tablets or capsules. These are also known to those skilled in the pharmaceutical art.

Treatment of patients suffering from C. difficile associated diseases with the combination of two active ingredients can take place not only concurrently in a single or separate dosage form but also following administration of one ingredient with the other. Preferably, administration of the inventive IgA is followed by administration of the antibiotic.

The antibody of the present invention is contained in secretory IgA provided to a subject suffering C. difficile infection or symptoms thereof: In such form, the amount of secretory IgA provided to the patient is about 1 gram per day. Typically amounts from about 0.1 to 50 grams per day will be used and preferably, 1 to 10 grams per day. For example, about 1 to 2 grams of secretory IgA could be given to a subject 3 to 4 times per day. The doses of the secretory IdA antibody formulation to be administered will depend upon the subject and the subject's medical history. Dosages of the specific secretory IgA for adult humans envisioned by the present invention and considered to be therapeutically effective will range from between about 0.1 to 500 mg. However, it is to be understood that doses can readily be adjusted to provide appropriate amounts of the secretory IgA antibody to any subject, including children.

The invention is further described by reference to the following detailed examples, wherein the methodologies are as described below. These examples are not meant to limit the scope of the invention that has been set forth in the foregoing description. Variations within the concepts of the invention are apparent to those skilled in the art.

### EXAMPLE 1

Polyclonal IgA is obtained from pooled human plasma following Cohn cold ethanol fractionation to produce fraction III precipitate. IgA is further purified by adsorption onto an ion exchange medium in neutral or slightly acidic conditions. Alternatively, monoclonal IgA is obtained from an IgA-producing hybridoma. The IgA is then coupled to recombinant J chains by disulfide bonding which is accomplished in mildly oxidizing conditions. The molar ratio of JgA to J chain is 2:1 or 3:1. IgA-J chain conjugates are purified. IgA-J chain conjugates are then further coupled to recombinant secretory component again by disulfide bonding in mildly oxidizing conditions, preferably at a molar ratio of secretory component to IgA-J chain conjugates of 1:1. IgA containing both J chain and secretory component is again purified. Purified IgA containing J chain and secretory component is stabilized by the addition of human serum albumin to a final concentration of 5%. The final solution is adjusted to a therapeutic dose of 5 mg IgA.

An ELISA assay will be used to confirm that the IgA preparation contains specific anti-C. difficile IgA.

ELISA Method

Human secretory IgA levels to C. difficile is measured by ELISA using a modification of the method previously described (C. P. Kelly et al., Gastroenterology 1992; 102:35-40; D. Y. M. Leung et al., J. Pediatr. 1991; 118:633-637 and Bacon and Fekety. Diagn. Microbiol. Infect. Dis. 1994; 18:205-209). Coating antigens used to measure IgA titers included purified C. difficile toxin A and purified C. difficile toxin.

Toxigenic Clostridium difficile is cultured for 72 hours in brain heart infusion broth (Beckton Dickinson, Cockeysville, Md.). The conditioned medium is centrifuged and the supernatant filter sterilized by passage through a 45 um filter (Nalgene). C. difficile toxins A and B are purified from the broth culture supernatant as previously described (C. Pothoulakis et al., J. Clin. Invest. 1991; 88:119-125).

Microtiter plates (Immulon II, Dynatech) are coated with C. difficile toxin A or toxin B (each at 10 μg protein per ml in carbonate buffer pH 9.6, 100 μl per well) by incubation for 2 hours at 37° C. followed by overnight incubation at 4° C. Plates are washed between each incubation step using phosphate buffered saline with 0.05% Tween 20 (PBS-T). Plates are then blocked with 2% human serum albumin (ICN, 100 μl/well) in PBS and incubated for 1 hour at room temperature.

All assays are performed in triplicate.

Horseradish peroxidase-labeled goat anti-human IgA (catalog number STAR92P, AbD Serotec) is used as the secondary antibody (0.2 ug/ml in PBS with 2% human serum albumin) incubated for one hour at 37° C. TMB microwell peroxidase substrate (KPL Laboratories) is used as substrate (100 μl/well) and stopped after 2 to 5 minutes with an equal volume of 1 M phosphoric acid. The optical density is then read at 450 nm with 630 nm as reference using an automated photometer (Dynatech). Controls include substitution of the secondary antibody with peroxidase labeled anti-murine IgA and omission of the peroxidase substrate solution. Results are expressed at the mean optical density of test wells minus mean optical density of background wells (coated with human serum albumin alone).

### EXAMPLE 2

To demonstrate that secretory IgA is capable of inhibiting the enterotoxic effects of C. difficile toxins.

Enterotoxicity Method

Fasting male Wistar rats are anesthetized by intraperitoneal injection of sodium pentobarbital. Laparotomy is performed, the renal pedicles tied and 3H-mannitol (10 μCi, PerkinElmer Life Sciences, Boston, Mass.) administered intravenously. Closed ileal loops (5 cm) are then formed and injected with 400 μl of 50 mM Tris buffer (pH 7.4) or with Tris buffer containing C. difficile culture filtrate (20 ug of protein). The inhibitory effect of secretory IgA is assessed by the addition of secretory IgA (200 ug) to the toxins prior to injection into the ileal lumen.

The abdominal incision is closed and anesthesia maintained with sodium pentobarbital. The animals are sacrificed after 4 hours and the ileal loops immediately harvested. Loop weight to length ratio is determined as a measure of enterotoxin effect. Mannitol excretion, indicating intestinal permeability, is measured by counting radioactivity in the luminal fluid. Ileal tissue samples are also fixed in formalin, paraffin-embedded and sections stained with hematoxylin and eosin. The histologic severity of enteritis is graded taking into account the following features: i) neutrophil margination and tissue infiltration, ii) hemorrhagic congestion and edema of the mucosa, iii) epithelial cell damage. A score of 0 to 3 denotes increasingly severe pathological changes.

### EXAMPLE 3

Treatment of a Person Ill with C. difficile Associated Disease with Secretory IgA

An adult individual ill with C. difficile associated disease is treated with secretory IgA containing antibody activity against C. difficile toxin. Treatment is with 1 gram orally three times daily together with vancomycin in appropriate dosage. Treatment is continued until symptoms resolve and the stool becomes negative for C. difficile toxin.

### REFERENCES

Bacon A. E. 3rd, Fekety R. lrnrunoglobulin G directed against toxins A and B of Clostridium difficile in file general population and patients with antibiotic-associated diarrhea. Diagn. Microbiol. Infect. Dis, 1994; 18:205-209.

Barroso L. A., Wang S. Z., Phelps C. J., Johnson J. L., Wilkins T. D. Nucleotide sequence of Clostridium difficile toxin B gene. Nucleic Acids Res. 1990; 18:4004.
Berzofsky J. A., Berkower I. J., Epstein S. L., Monoclonal Antibodies in Chapter 12, Antigen-Antibody Interactions and Monoclonal Antibodies, pp. 455-465 in Fundamental Immunology, Third Edition, W. E. Paul (ed), Raven Press, NY 1993. Berzofsky et al., Fundamental Immunology, Third Edition, 1993, pp 455-462.
Boesman-Finkelstein M., Walton N. E., Finlcelstein R. A. Bovine lactogenic immunity against cholera toxin-related enterotoxins and Vibrio cholerae outer membranes. Infect. Immun. 1989; 57:1227-1234.
Brussow H., Hilpert H., Walther I., Sidoti J., Mietens C., Bachmann P. Bovine milk immunoglobulins for passive immunity to infantile rotavirus gastroenteritis. J. Clin. Microbiol. 1987; 25:982-986.
Colm E. J., Strong L. E., Hughes W. L., Jr., Mulford D. J., Ashworth J. N., Melin M., Taylor H. L., Preparation and Properties of Serum and Plasma Proteins IV. A System for the Separation into Fractions of the Protein and Lipoprotein Components of Biological Tissues and Fluids, J. Am. Chem. Soc. 1946; 68; 459-475.
Cone L. A., Lopez C., Tarleton H. L., Jodoin D., Taylor M., Gade-Andavolu R., Dreisbach L. P. A durable response to relapsing Clostridium difficile colitis may require combined therapy with higlh-dose oral vancomycin and intravenous immune globulin. Infect Dis Clin Pract 2006; 14:217-220.
Corthesy B., Recombinant Secretory Id for Immune Intervention Against Mucosal Pathogens, Biochem. Soc. Trans. 1997, 25; 471-475.
Corthier et al., Emergence in Gnotobiotic Mice of Nontoxinogenic Clones of clostridium difficile from a Toxinogenic One, Infection and Immunity, June. 1988, pp. 1500-1504.
Cortliier et al., Protection Against Experimental Pseudomembranous Colitis in Gnotobiotic Mice by Use of Monoclonal Antibodies Against clostridium difficile Toxin A, Infection and Immunity, March. 1991, pp. 1192-1195.
Crottet P., Cottet S., Corthesy B., Expression, Purification and Biochemical Characterization of Recombinant Murine Secretory Component, A Novel Tool in Mucosal Immunology, Biochem. J. 1999, 341; 299-306.
Dove C. H., Wang S. Z., Price S. B., Phelps C. J., Lyerly D. M., Wilkins T. D. and Johnson J. L.; Lyerly et al. Molecular characterization of the Clostridium difficile toxin A gene. Infect. Immun. 1990; 58:480-488.
Ehrich et al., Production of clostridium difficile Antitoxin, Infection and Immunity, June. 1980, pp. 1041-1043.
Fayer R., Guidry A., Blagburn B. L. Immunotherapeutic efficacy of bovine colostral immunoglobulins from a hyperimmunized cow against cryptosporidiosis in neonatal mice. Infect. Immun., 1990; 58:2962-2965.
Gerding et al., clostridium difficile—Associated Diarrhea, Archives of Internal Medicine, vol. 146, January. 1986, pp. 95-100.
Hilpert H., Brussow H., Mietens C., Sidoti J,, Lerner L., Werchau H. Use of bovine milk concentrate containing antibody to rotavirus to treat rotavirus gastroenteritis in infants. J. Infect. Dis. 1987; 156:158-166.
Johnson S. et al. Infect. Immun. 1995; 63:3166-3173,
Jones R. M. L., Schweikart F., Frutiger S., Jaton J-C., Hughes G. J. Thiol-disulfide redox buffers maintain a structure of immunoglobulin A that is essential for optimal in vitro binding to secretory component. Biochimica et Biophysica Acta 1998; 1429:265-274.
Kelly et al., clostridium difficile Colitis, New England Journal of Medicine, vol. 330, January. 1994, pp. 257-262.
Kelly et al., Human Colonic Aspirates Containing Immunoglobulin A Antibody to clostridium difficile Toxin A Wiibit Toxin A—Receptor Binding, Gastroenterology, vol. 102, No. 1, pp. 35-40.
Kohler G., Milstein C., Continuous Cultures of Fused Cells Secreting Antibody of Predetermined Specificity, Nature 1975; 256; 495-497.
Leung D. Y., Kelly C. P., Boguniewicz M., Potloulakis C., LaMont J. T., Flores A. Treatment with intravenously administered gamma globulin of chronic relapsing colitis induced by Clostridium difficile toxin. J. Pediatr. 1991 April; 118(4(Pt 1)):633-637.
Libby J. M., Jortner B. S., Wilkins T. D. Effects of the two toxins of Clostridium difficile in antibiotic-associated cecitis in hamsters. Infect. Immun. 1982 May; 36(2):822-829.
Lima et al., Effects of clostridium difficile Toxins A and B in Rabbit Small and Large Intestine In Vivo and on Cultured Cells In Vitro, Infection and Immunity, March. 1988, pp. 582-588.
Louie T. J., Peppe J., Watt C. K., Johnson D., Mohammed R., Dow G., Weiss K., Simon S., John J. F. Jr., Garber G., Chasan-Taber S., Davidson D. M.; Tolevamer Study Investigator Group. Tolevarner, a novel nonantibiotic polymer, compared with vancomycin in the treatment of mild to moderately severe Clostridium difficile-associated diarrhea. Clin. Infect. Dis. 2006; 43:411-20.
Lullau E., Heyse S., Vogel H., Marison I., von Stockar U., Kraehlanbuhl J-P., Corthesy B., Antigen Binding Properties of Purified Immunoglulin A Antibodies, J. Biol. Chem. 1996; 271:16300-16309.
Lyerly D. M., Krivan H. C., Wilkins T. D. Clostridium difficile: its disease and toxins. Clin. Microbiol. Rev. 1988; 1:1-18.
Lyerly D. M., Phelps C. J., Toth J., Wilkins T. D. Characterization of toxins A and B of Clostridium difficile with monoclonal antibodies. Infect. Immun. 1986; 54:70-76.
Lyerly D. M., Bostwick E. F., Binion S. B., Wilkins T. D. Passive immunization of hamsters against disease caused by Clostridium difficile by use of bovine immunoglobulin G concentrate. Infect. Immun. 1991; 59:2215-2218.
Lyerly D. M., Lockwood D. E., Richardson S. H., Wilkins T. D. Biological activities of toxins A and B of Clostridium difficile, Infect. Immun. 1982; 35:1147-1150.
Lyerly D. M., Saum K. E., MacDonald D. K., Wilkins T. D. Effects of Clostridium difficile toxins given intragastrically to animals. Infect. Immun. 1985; 47:349-352.
Mahe et al., Effect of Various Diets on Toxin Production by Two Strains of clostridium difficile in Gnotobiotic Mice. Infection and Immunity, August. 1987, pp. 1801-1805.
Martinez et al., Purification and Characterization of clostridium sordellii Hemorrhagic Toxin and Cross-Reactivity with clostridium difficile Toxin A (Enterotoxin), Infection and Immunity, May 1988, pp. 12-15-1221.
McFarland et al., Nosocomial Acquisition of clostridium difficile Infection, The New England Journal of Medicine, January. 1989, pp. 204-210.
McFarland et al., Review of clostridium difficile Associated Diseases, American Journal of Infection Control, vol. 14, No. 3, June. 1986, pp. 99-104.
McPherson S., Rees C. J., Ellis R., Soo S. and Panter S. J. Intravenous Immunoglobulin for the Treatment of Severe, Refractory, and Recurrent Clostridium difficile Diarrhea. Diseases of the Colon & Rectum, 2006; 49(5):640-645.
Med. Letter Drugs Ther. 2006; 48:89-90,92.
Mietens C., Keinhorst H., Hilpert H., Gerber H., Amster H., Pahud J. J. Treatment of infantile E. coli gastroenteritis with specific bovine anti-E. coli milk immunoglobulins. Eur. J. Pediatr. 1979; 132:239-252.
Mitchell et al., Effect of Toxin A and B of clostridium difficile on Rabbit Ileum and Colon, Gut, 1986, vol. 27, pp. 78-85.
Morris et al., Role of Surgery in Antibiotic-Induced Pseudomembranous Enterocolitis, The American Journal of Surgery, vol. 160, November. 1990, pp. 535-539.
Oncley J. L., Melin M., Richert D. A., Cameron J. W., Gross P. M., Jr., The Separation of the Antibodies, Isoagglutinins, Prothrombin, Plasminogen and β1-Lipoprotein into Subfractions of Human Plasma. J. Am. Chem. Soc. 1949; 71:541-550.
Pothoulakis C., LaMont J. T., Eglow R., Gao N., Rubins J. B., Theoharides T. C., Dickey B. F. Characterization of rabbit ileal receptors for Clostridium difficile toxin A. Evidence for a receptor-coupled G protein. J. Clin. Invest. 1991; 88:119-25.
Rotlhman et al., Differential Cytotoxic Effects of Toxins A and B Isolated from clostridium difficile, Infection and Immunity, November. 1984, pp. 324-331.
Salcedo J. et. al. Gut 1997; 41:366-370.
Strong L. E., Blood Fractionation, pp. 576-602 in vol. 3, Kirk-Othmer Encyclopedia of Chemical Techinology. Second Edition, H. F. Mark, J. J. MelCetta, D. F. Othnmer (eds), Interscience Publishers, NY 1963, pp. 576-602.
Stubbe H. et al. J. Immunol. 2000; 164:1952-1960.
Symersky J., Novak J., McPherson D. T., DeLucas L., Mestecky J. Expression of the recombinant human immunoglobulin J chain in Escherichia coli. Mol. Immunol. 2000; 37:133-140.
Tacket C. O., Losonsky G., Link H., Hoang Y., Guesry P., Hilpert H., Levine M. M. Protection by milk immunoglobulin concentrate against oral challenge with enterotoxigenic Escherichia coli. N. Engl. J. Med. 1988; 318:1240-3.
Tjellstrom B., Stenhammar L., Eriksson S., Magnusson K. E. Oral immunoglobulin A supplement in treatment of Clostridium difficile enteritis. Lancet 1993; 341(8846):701-702.
Triadafilopoulos et al., Differential Effects of clostridium difficile Toxins A and B on Rabbit Ileum, Gastroenterology, 1987, vol. 93, pp. 273-279.
Tucker et al., Toxin A of clostridium difficile Is a Potent Cytotoxin, Journal of Clinical Microbiology, May 1990, pp. 869-871.
Weltzin R., Traina-Dorge V., Soike K., Zhang J. Y., Mack P., Soman G., Drabik G., Monath T. P., Intranasal Monoclonal IgA Antibody against Respiratory Syncytial Virus Protects Rhesus Monkeys against Upper and Lower Respiratory Tract Infection. J. Infect. Dis. 1996; 174:256-261.
Weltzin R., Hsu S. A., Mittler E. S., Georgakopoulas K., Monath T. P., Intranasal Monoclonal Immunoglobulin A against Respiratory Synctial Virus Protects against Upper and Lower Respiratory Tract Infections in Mice. Antimicrob. Agents Chemother. 1994; 38:2785-2791.
Wilcox M. H. J. Antimicrob. Chemoth. 2004; 53:882-884.
Yoshiyama Y., Brown W. R. Specific antibodies to cholera toxin in rabbit milk are protective against Vibrio cholerae-induced intestinal secretion. Immunology. 1987; 61:543-547.

Patent applications and publications mentioned in the specification are indicative of the levels of those skilled in the art to which the invention pertains. These applications and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A composition for treating C. difficile associated disease in a human comprising:
administering to said human suffering therefrom an amount of polyclonal monomeric IgA combined with a recombinant J chain to form an IgA-J chain conjugate in a molar ratio of the IgA to the J chain of 2:1 or greater and combined with a recombinant secretory component in a molar ratio of an IgA-J chain conjugate to the secretory component of 1:1 forming a dimeric or polymeric IgA;
formulating agents combined with the dimeric or polymeric IgA as the composition for treating C. difficile associated disease in a dosing form selected from the group consisting of: a solid oral dosing form, a liquid oral dosing form, and a suppository.

2. The composition of claim 1 wherein the solid oral dosing form is a tablet or a capsule and further comprises an enteric coating on the tablet or the capsule.

3. The composition of claim 1, wherein the secretory IgA is microencapsulated.

4. The composition of claim 1 wherein the dimeric or polymeric IgA is provided in the dosing form in an amount of between 0.1 and 50 grams.

5. The composition of claim 1 further comprising an antibiotic present in a therapeutically effective amount in the dosing form.

6. The composition of claim 1 wherein the subject is human and the recombinant J chain is human.

7. The composition of claim 1 wherein the IGA-J chain conjugate is combined with to the recombinant secretory component by a disulfide linkage.

8. A process for manufacturing a medicament for the treatment of C. difficile associated disease in a human comprising:
collecting polyclonal monomeric IgA as a byproduct of cold ethanol fractionation of pooled plasma derived from more than one human individual;
subjecting the polyclonal monomeric IgA to antiviral treatment to yield a virus free polyclonal monomeric IgA;
sterilizing the virus free polyclonal monomeric IgA to yield sterile polyclonal monomeric IgA;
sequentially modifying the sterile polyclonal monomeric IgA with J chain and secretory component to form dimeric or polymeric IgA; and
mixing the dimeric or polymeric Ilg with formulating agents in a dosing form selected from the group consisting of: a solid oral dosing form, a liquid oral dosing form, and a suppository.

9. The process of claim 8 further comprising adding an enteric coating to the dosing form.

10. The process of claim 8 further comprising microencapsulating the dimeric or polymeric IgA.

11. The composition according to claim 8, wherein the pooled plasma is derived from specifically immune or immunized donors.

12. A method of treating C. difficile associated disease in a subject comprising: administering to the subject the composition of claim 1.

13. The method of claim 12, wherein the composition of claim 1 is administered at least once daily and in an amount of the dimeric or polymeric IgA of between 0.1 and 50 grams per day.

14. The method of claim 12 further comprising:
providing the human with a therapeutically effective amount of an antibiotic selected from the group consisting of: vancomycin and metronidazole.

15. The method of claim 14 wherein the antibiotic is provided simultaneously with the dimeric or polymeric IgA.

16. The method of claim 14 wherein both vancomycin and metronidazole are provided to the human.

17. The method of claim 14 wherein the antibiotic is provided and discontinued prior to the administrating of the composition of claim 1.

18. A process for manufacturing a medicament for the treatment of C. difficile associated disease in a human comprising:
producing monoclonal monomeric IgA by hybridoma technique;
sequentially modifying the monoclonal monomeric IgA with J chain and secretory component to form dimeric or polymeric IgA; and
mixing the dimeric or polymeric IgA with formulating agents in a dosing form selected from the group consisting of: a capsule, tablet, and a suppository.

19. The process of claim 18 furtler comprising adding an enteric coating to the dosing form.

20. The process of claim 19 further comprising microencapsulating the dimeric or polymeric IgA.

21. The composition according to claim 18, wherein the pooled plasma is derived from specifically immune or immunized donors.
